(19) United States
(12) Patent Application Publication
(10) Pub. No.: US 2024/0315781 A1
(43) Pub. Date: Sep. 26, 2024

(54) DEFLECTABLE SENSING INSTRUMENT SYSTEMS AND METHODS

(71) Applicant: INTUITIVE SURGICAL OPERATIONS, INC., Sunnyvale, CA (US)

(72) Inventors: Lucas S. Gordon, San Jose, CA (US); Joseph D. Bogusky, San Jose, CA (US); Samuel Raybin, San Jose, CA (US); Serena H. Wong, Los Altos, CA (US)

(21) Appl. No.: 18/611,485

(22) Filed: Mar. 20, 2024

Related U.S. Application Data
(60) Provisional application No. 63/491,386, filed on Mar. 21, 2023.

(51) Int. Cl. A61B 34/20; A61B 10/04; A61B 18/00; A61B 18/14; A61B 34/30; A61M 25/01; A61M 25/06

(52) U.S. Cl. CPC A61B 34/20; A61B 10/04; A61B 18/1492; A61B 2010/045; A61B 2018/00577; A61B 2018/00613; A61B 2034/2051; A61B 2034/2055; A61B 2034/2061; A61B 2034/2063; A61B 2034/2065; A61B 2034/301; A61M 25/0138; A61M 25/0147; A61M 2025/0681

(57) ABSTRACT

A system may comprise a sensing instrument including an elongate flexible member with a channel extending therein. The elongate flexible member may include a proximal portion, a distal portion, and a flexure portion between the distal and proximal portions. The sensing instrument may also include a flexure at the flexure portion of the elongate flexible member, a sensing element coupled at the distal portion of the elongate flexible member, and a flexure control apparatus extending within the elongate flexible member and configured to bend the flexure to change an orientation of the sensing element relative to the proximal portion of the elongate flexible member. The sensing instrument may also include an exit port in the proximal portion of the elongate flexible member.

### CROSS-REFERENCED APPLICATIONS

This application claims priority to and benefit of U.S. Provisional Application No. 63/491,386 filed Mar. 21, 2023 and entitled “Deflectable Sensing Instrument Systems and Methods,” which is incorporated by reference herein in its entirety.

### FIELD

The present disclosure is directed to systems and methods for deflecting a sensing portion of an instrument during a sensor-guided procedure.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools include instruments such as therapeutic, diagnostic, biopsy, and surgical instruments. Medical tools may be inserted into anatomic passageways and navigated toward a region of interest within a patient anatomy. Navigation may be assisted using optical or ultrasound images of the anatomic passageways and surrounding anatomy, obtained pre-operatively and/or intra-operatively. Intra-operative imaging of an interventional tool by an imaging probe or catheter through which the interventional tool is inserted may provide improved navigational guidance and confirmation of engagement of the tool with the target tissue. Improved systems and methods are needed for positioning the imaging probe to clearly visualize the target tissue and the interventional tool during a procedure.

### SUMMARY

Consistent with some embodiments, a system may comprise a sensing instrument including an elongate flexible member with a channel extending therein. The elongate flexible member may include a proximal portion, a distal portion, and a flexure portion between the distal and proximal portions. The sensing instrument may also include a flexure at the flexure portion of the elongate flexible member, a sensing element coupled at the distal portion of the elongate flexible member, and a flexure control apparatus extending within the elongate flexible member and configured to bend the flexure to change an orientation of the sensing element relative to the proximal portion of the elongate flexible member. The sensing instrument may also include an exit port in the proximal portion of the elongate flexible member. Other embodiments include corresponding computer systems, apparatus, and computer programs recorded on one or more computer storage devices, each configured to perform the actions of the methods.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 illustrates an example of a medical instrument system in a patient anatomy near a target tissue, according to some examples.

FIG. 2 illustrates a medical instrument system extended within an anatomic passage, according to some examples.

FIG. 3 illustrates an elongated medical instrument system including a delivery catheter and a sensing instrument, according to some examples.

FIG. 4 illustrates the medical instrument system of FIG. 3 extending within an anatomic passage.

FIG. 5 illustrates a pulmonary lymph node system.

FIG. 6 illustrates a medical instrument system extending within an anatomic passage, according to some examples.

FIG. 7 illustrates a medical instrument system extending within an anatomic passage, according to some examples.

FIG. 8 illustrates a cross sectional view of a sensing instrument, according to some examples.

FIG. 9 illustrates a cross sectional view of a sensing instrument, according to some examples.

FIG. 10 illustrates a distal end portion of an integrated sensing instrument and delivery catheter, according to some examples.

FIG. 11A illustrates a flexure portion of a sensing instrument, according to some examples.

FIG. 11B illustrates a flexure portion of a sensing instrument, according to some examples.

FIGS. 12A and 12B illustrates a flexure portion of a sensing instrument, according to some examples.

FIG. 13 illustrates a side view of a control apparatus actuation system, according to some examples.

FIG. 14 illustrates a partially exploded view of an actuator device, according to some examples.

FIG. 15 illustrates a cross-sectional view of the actuator device of FIG. 14.

FIG. 16 is a flowchart illustrating a method for deflecting a sensing instrument during a sensor-guided procedure.

FIG. 17 illustrates a simplified diagram of a robot-assisted medical system according to some examples of the present disclosure.

FIGS. 18A and 18B are simplified diagrams of a medical instrument system according to some examples.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

The techniques disclosed in this document may be used to enhance intra-operative sensing instruments, including intra-operative imaging instruments, and their use in minimally invasive procedures. In some examples, intra-operative sensing data, including imaging data, May be utilized to verify real-time accurate placement of a treatment or diagnostic tool within an anatomical target during a medical procedure. For example, a sensing instrument may be used to provide direct visual guidance of a tool as the tool is advanced toward a target. The sensing instrument may include a sensing element and a flexure portion deflectable to change a position and/or orientation of the sensing element to achieve contact with the patient anatomy. Although some of the sensing instruments described herein are ultrasound imaging instruments, it is contemplated that the systems and methods described herein may be applied to other imaging and sensing modalities without departing from the scope of the present disclosure.

The systems and techniques described in this document may be used in a variety of medical procedures that may improve accuracy and outcomes through use of intra-operative imaging. For example, intra-operative imaging may be used to biopsy lesions or other tissue to, for example, evaluate the presence or extent of diseases such as cancer or surveil transplanted organs. As another example, intra-operative imaging may be used in cancer staging to determine via biopsy whether the disease has spread to lymph nodes. The medical procedure may be performed using hand-held or otherwise manually controlled imaging probes and tools (e.g., a bronchoscope). In other examples, the described imaging probes and tools many be manipulated with a robot-assisted medical system.

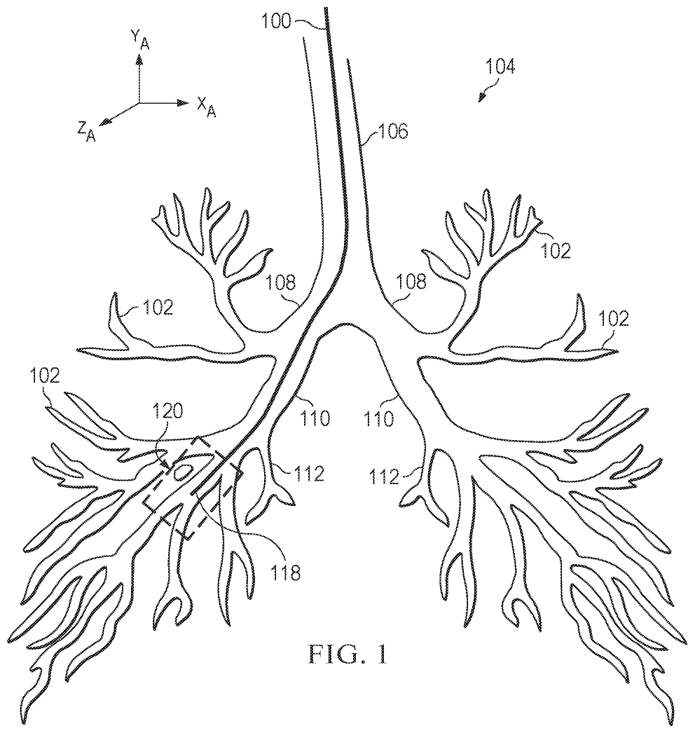

FIG. 1 illustrates an elongated medical instrument system 100 extending within branched anatomic passageways or airways 102 of an anatomical structure 104. In some examples the anatomic structure 104 may be a lung and the passageways 102 may include the trachea 106, primary bronchi 108, secondary bronchi 110, and tertiary bronchi 112. The anatomic structure 104 has an anatomical frame of reference (X_{A}, Y_{A}, Z_{A}). A distal end portion 118 of the medical instrument system 100 may be advanced into an anatomic opening (e.g., a patient mouth) and through the anatomic passageways 102 to perform a medical procedure, such as a biopsy, at or near a target tissue 113.

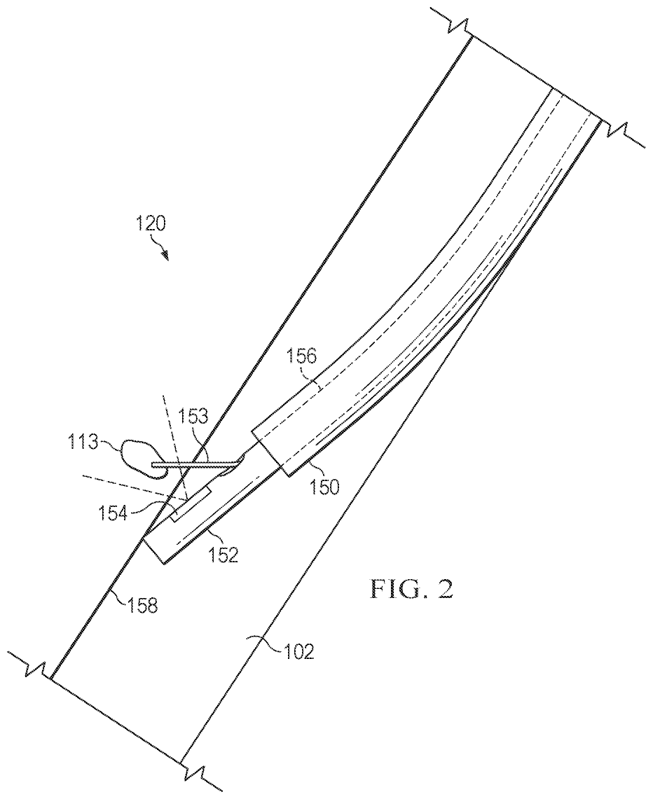

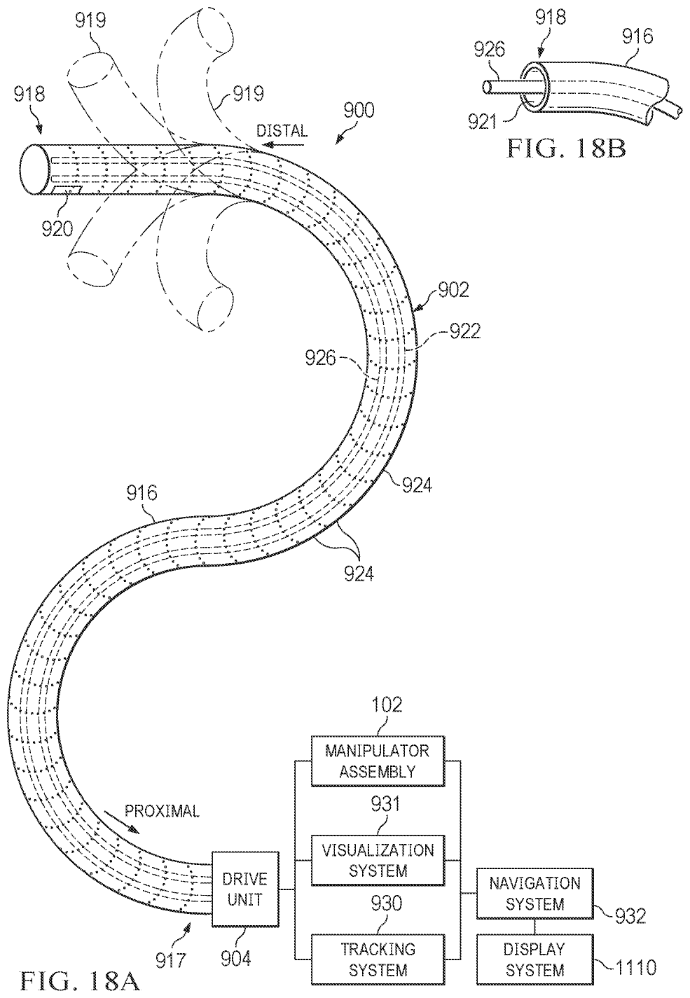

As shown in FIG. 2 an elongated medical instrument system (e.g., the elongated medical instrument system 100) may include a delivery catheter 150 for delivering a sensing instrument 152 into the anatomic passageway 102. In some examples, the delivery catheter may be a bronchoscope or a robot-assisted steerable catheter system. An example of a delivery catheter system that is bendable and steerable in multiple degrees of freedom is described below in FIGS. 18A and 18B (e.g. system 900).

The sensing instrument 152 may include a sensing element 154. In some examples, the sensing element 154 may include one or more imaging elements, such as an ultrasound transducer or a visible light optical imaging element. Ultrasound transducers may include transducer arrays that may be comprised of a plurality of transducers of any size or shape including circles, rectangles, moon, etc. In other examples, the sensing element may be an infrared sensor, a three-dimensional tomography sensor, or another type of sensor that gathers information about the surrounding tissue. The sensed information about the surrounding tissue may include the location of the target tissue 113 relative to the sensing instrument 152 and/or an interventional tool 153. The location of the target tissue 113 may be useful in positioning the sensing instrument 152 so that the interventional tool 153, such as a biopsy tool (e.g., needle, forceps, etc.), extendable from the instrument 152 may contact the target tissue. The sensing instrument 152 may be slidable through a channel 156 (also referred to as working channel) of the delivery catheter 150 and extendable from a distal end portion 151 of the delivery catheter 150. The sensing instrument 152 may also be withdrawn proximally and removed from the delivery catheter 150.

For many sensing modalities, including ultrasound imaging, contact between the sensing element 154 and a wall 158 of the anatomic passageway 102 may be required or may improve the quality of the sensor or imaging data received from the sensing element. For example, if the sensing element is an ultrasound transducer, contact between the ultrasound transducer (or a protective covering thereof) and the wall 158 may eliminate air gaps and promote the effective transmission of the ultrasound signal and the generation of a clear image. Sensing instruments that include a deflectable sensing element may be more adaptable to a variety of anatomic passageway configurations and target tissue locations. For example, a deflectable sensing element may be useful in an anatomic passageway that is larger than a diameter of the sensing instrument, as in FIG. 1. In such an environment, contact between the distal portion of the sensing instrument and the wall of the anatomic passageway may be difficult to ensure because, without a snug fit, the distal end portion may be angled or distanced away from the wall. Furthermore, a sensing element that is deflectable with respect to a more proximal exit port for an interventional tool may provide greater versatility for dual operations (e.g., imaging and operating the interventional tool) with the sensing instrument. For example, an ultrasound transducer at the distal end portion of a sensing instrument that is deflectable relative to a biopsy instrument extendable from the sensing instrument may be configured for both a preferred orientation of the sensing element (e.g., in contact with the passageway wall to eliminate an air gap) and also a preferred orientation of the biopsy instrument (e.g., along a trajectory that intersects the target tissue). Control of the deflection of the sensing element may be independent of other navigation articulation or steering control components, including pull wires, control cables, or other articulation or steering control components of the sensing instrument or of the delivery catheter.

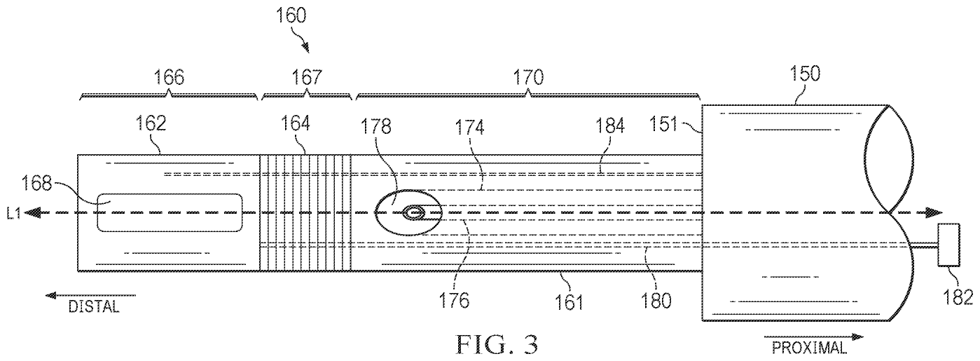

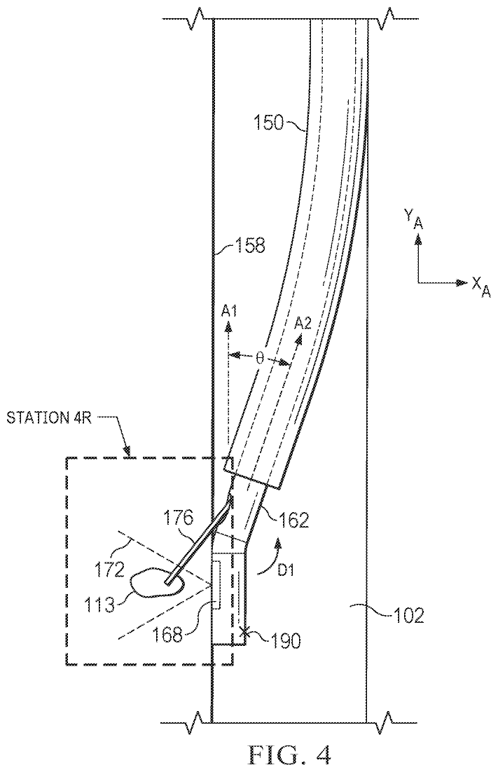

FIGS. 3 and 4 illustrate a sensing instrument with a deflectable sensing element. More specifically, FIGS. 3 and 4 illustrate an elongated medical instrument system 160 including the delivery catheter 150 and a sensing instrument 162 that is deflectable to engage the wall 158 of the anatomic passageway 102. The sensing instrument 162 may include an elongate flexible member 161 including a distal portion 166 that is extendable distally from the catheter 150. The sensing instrument 162 may extend along a longitudinal axis L1. A flexure portion 167 extending between the distal portion 166 and a proximal portion 170 may include a flexure 164 that allows the distal portion 166 to deflect to engage the passageway wall 158. In this example, a sensing element 168, which may include an ultrasound transducer, is located at the distal portion 166 of the sensing instrument 162, distal of the flexure 164, and has a sensing field 172. The ultrasound transducer may include a forward-facing ultrasound array (comprising one or more transducers, receivers, and/or emitters) configured to capture images in an imaging field of view (e.g., the sensing field 172). The flexure 164 is bendable to allow the distal portion 166 to bend relative to a proximal portion 170 of the sensing instrument 162. For example, an angle θ may be formed between an axis A1 of the distal portion 166 and an axis A2 of the proximal portion 170 so that the sensing element 168 is flush with or otherwise in contact with the passageway wall 158. With the sensing element 168 in contact with the wall 158, the field of view 172 of the sensing element may clearly capture on image of the tissue adjacent to the wall 158, including the target tissue 113.

A flexure control apparatus 180 may include one or more control wires, tendons, or rods extending through the sensing instrument 162 to the distal portion 166 or to a distal area of the flexure portion 167. The flexure control apparatus 180 may be separate and independently operated from the cables, linkages, or other steering controls that controllably bend the delivery catheter 150. Further, the flexure control apparatus may articulate the distal portion 166 and the flexure 164 independently of any steering or articulation of the proximal portion 170. The control apparatus 180 may be pushed, pulled, or otherwise actuated (e.g., manually or with robot-assistance) by an actuator 182 to create the bend angle θ at the flexure 164 and to change an orientation of the sensing element 168 relative to the proximal portion 170 of the elongate flexible member 161. In some examples, the bend angle θ may be controllable or selectable in a range from 0 to 90 degrees. In some examples, the control apparatus 180 may be operable to bend the flexure to a predetermined bend angle θ. For example, the control apparatus may be operable to create a bend angle of approximately 30 degrees. The control apparatus for the deflection of the flexure 164 may be independent of control apparatus for the steering or articulation of the delivery catheter 150. A deflection sensor 184 may extend through the sensing instrument 162 to measure the deflection of the axis A1 relative the axis A2 which may correspond to the angle θ. In some examples, the deflection sensor may include, for example an optical fiber shape sensor or one or more electromagnetic (EM) sensors.

The sensing instrument 162 may also include a passage 174 through which an interventional tool 176 may be extended to emerge from an exit port 178 of the sensing instrument 162. In this example, the exit port 178 may be located in the proximal portion 170, proximal of the flexure 164, but in other examples, the exit port 178 may be in the distal end portion or in the flexure. The exit port 178 may extend through an outer surface 171 of the elongate flexible member 161. The interventional tool 176 may include, for example, a biopsy or tissue sampling tool (e.g., needle or forceps), an ablation tool including a heated or cryo-probe, an electroporation tool, a medication delivery device, a fiducial delivery device, or another type of diagnostic or therapeutic device. In some examples, the interventional tool may be used to deliver a device into or near the target tissue. For example, a radiopaque marker or a drug delivery implant may be delivered by the interventional tool. In some examples, the interventional tool may have a flexible shaft. The interventional tool may include control wires or other control apparatus to bend or steer the direction of the interventional tool. Since it may be beneficial to provide real time visualization of the interventional tool positioned within or near a target tissue, the interventional tool may be delivered within the imaging field of view of the sensing element 168 (e.g., an ultrasound imaging instrument) for direct visualization of the interventional tool into the target 113. In some examples, if the exit port is proximal of the flexure, the deflection sensor 184 may provide information about the location and orientation of the sensing field (e.g., the ultrasound field of view) relative to the exit port and the interventional tool that extends therethrough.

In some examples, the control apparatus 180 may include a single push-pull control wire that that is operable to bend the flexure 164 in a single plane. In some examples, the flexure 164 may be pre-bent so that advancement or retraction of the single control wire causes the flexure 164 and the distal portion 166 to bend in a single plane. In other examples, the control apparatus 180 may include a pair of control tendons operable to bend the flexure 164 in a single plane. For example, one control tendon of the pair may operate to cause bending of the flexure in a first direct (e.g., flexion of the flexure) and the other control tendon of the pair may operate to cause bending of the flexure in a second direction opposite to the first direction (e.g., extension of the flexure). In other examples, the control apparatus 180 may include two pairs of control tendons operable to bend the flexure 164 in multiple planes (e.g., along pitch and yaw axes). In some examples in which the flexure 164 is operable to bend in a single plane, the coupling location of the control apparatus 180 to the distal portion 166 or the flexure portion 167 may determine the orientation of the bend of the flexure. A coupling location 190 of the control apparatus 180 may be selected based upon the region of the anatomy in which the sensing instrument 162 may be used and the orientation of the sensing element 168 relative to the wall 158 of the anatomic passageway 102, as explained in further detail below.

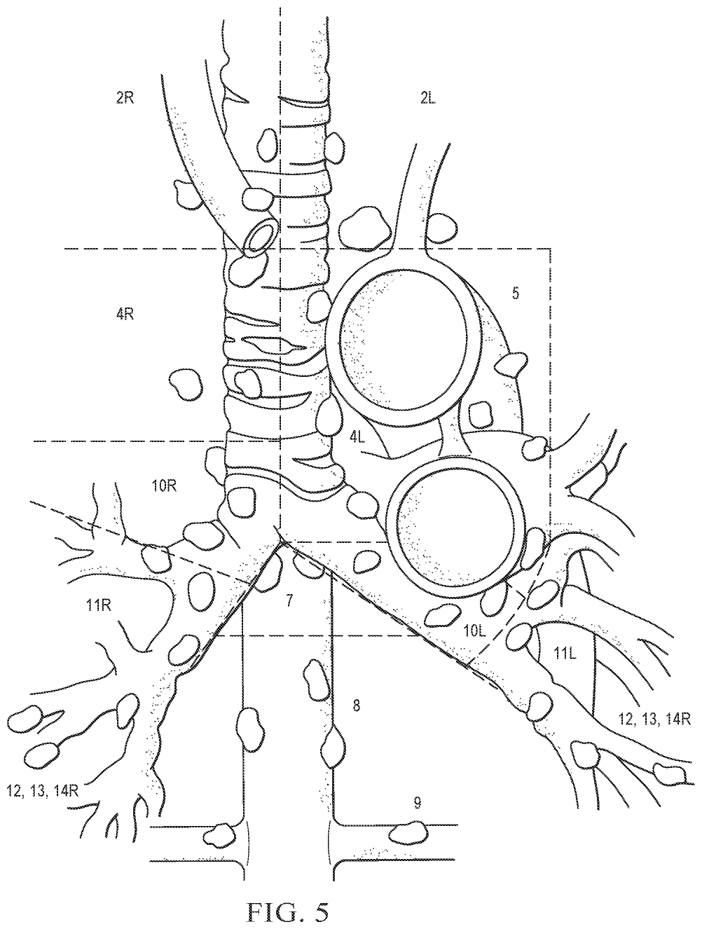

In some examples, the target tissue 113 may be a lymph node to be biopsied by the interventional tool 176. As shown in FIG. 5, the lymph nodes near the airways of the lungs may be located at predetermined regional node stations. For example, superior mediastinal nodes may be located along the trachea in upper and lower paratracheal regions at node stations 2R, 2L, 4R, 4L. Inferior mediastinal nodes may include lymph nodes located in subcarinal regional node station 7, para-esophogcal regional node station 8, and/or pulmonary ligament regional node station 9. Other lymph nodes may be adjacent to right and left bronchial passages branched from the trachea at regional node stations 10R, 10L. Interlobar lymph nodes may be located at station 11. Lobar lymph nodes may be located at regional node station 12. Segmental lymph nodes may be located at regional node station 13. Subsegmental lymph nodes may be located at regional node station 14.

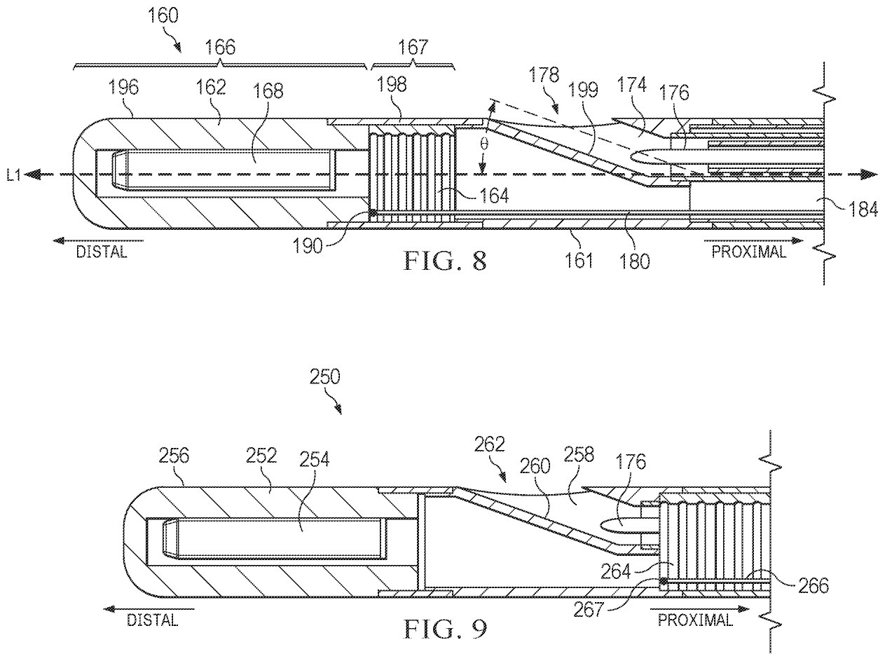

In the example of FIG. 4, target tissue 113 to be biopsied may be a right lower paratracheal lymph node located in station 4R. For a target tissue 113 in the region of station 4R, the delivery catheter 150 may be positioned against the wall 158 on an opposite side of the passageway 102 from the target tissue 113. The distal end portion 151 of the delivery catheter 150 may be steered or bent toward the target tissue 113. The sensing instrument 162 may be extended from the distal end portion 151 with the sensing element 168 rotated in the direction of the target tissue 113. In this example, the control apparatus 180, which may be a single control wire, may be coupled to or terminated at the distal portion 166 of the sensing instrument 162 at a location 190, opposite of the sensing element 168 or a transmission surface 196 (see FIG. 8) covering the sensing element. With the control apparatus 180 coupled at the location 190, actuation of the control apparatus 180 may cause the distal portion 166 to bend at the flexure 164 in the direction D1. The plane (e.g., the XAYA plane) of the bend angle θ may be consistent or predetermined relative to the sensing element 168 so that with the control apparatus 180 coupled at location 190, the sensing element 168 will predictably bend into generally parallel alignment and abutment with the wall 158. In some examples the bend angle θ may be approximately 30 degrees. Generally, if a control wire or tendon is coupled to the distal portion 166 on the transmission surface side of the axis L1, the flexure 164 may bend toward the direction of the transmission surface side. If a control wire or tendon is coupled to the distal portion 166 on an opposite side of the axis L1, the flexure 164 may bend in the direction opposite the transmission surface side. In some situations, a similar deflection may be achieved by bending the delivery catheter 150, but the force required to deflect the distal portion 166 may be less than a force required to actuate a delivery catheter to achieve a similar deflection.

As compared to the undeflectable sensing instrument 152 in FIG. 2, the deflected sensing instrument 162 with the flexure 164 may allow an ultrasound transducer to be positioned against an airway wall without an air gap between the wall and the transmission surface 196 of the sensing element 168. An air gap may degrade or render an image unusable. In the example of FIG. 2 that includes the undeflectable sensing instrument, it may not be possible to move the sensing element against the wall while still allowing the extension of the interventional tool to reach the target tissue. A deflectable sensing instrument 162 may allow for both a preferred orientation of the sensing element (e.g., in contact with the passageway wall to eliminate an air gap) and also a preferred orientation of the biopsy instrument (e.g., along a trajectory that intersects the target tissue).

With the transmission surface 196 engaged with the airway wall 158, the resulting ultrasound images may clearly show the extended interventional tool 176 and its proximity to the target tissue 113. The image may also clearly display the surrounding vasculature to be avoided by the interventional tool 176. The deflectable sensing instrument 162 may be particularly suited for anatomic passageways that are larger in diameter than the instrument diameter because achieving close contact between the transmission surface may be more difficult in larger passageways, without a deflectable distal portion. For example, an instrument with a diameter of 6 mm positioned within a 11-22 mm passageway may achieve improved imaging by deflection of the distal end portion. A deflectable distal portion may be particularly useful for imaging lymph nodes at stations (e.g., node station 4R, 4L, 7, 11) that may require complex instrument bending to align the sensing element with the node.

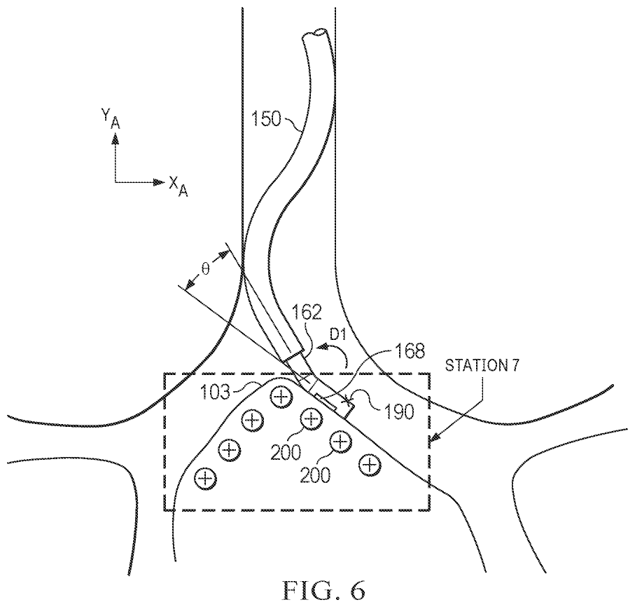

In the example of FIG. 6, a target tissue 200 to be biopsied may include one of a plurality of lymph nodes in the subcarinal region of station 7. To access the target tissue 200 in the region of station 7, the delivery catheter 150 may be positioned against the anatomic wall 158 on an opposite side of carina 103 from the target tissue 200. The distal end portion 151 of the delivery catheter 150 may be steered or bent toward the target tissue 200. The sensing instrument 162 may be extended from the distal end portion 151 with the sensing element 168 rotated in the direction of the target tissue 200. In this example, the control apparatus 180, which may be a single control wire, may be coupled to or terminated at the distal portion 166 or a distal area of the flexure portion 167 of the sensing instrument 162 at a location 190, opposite of the sensing element 168 or a transmission surface 196 of the sensing instrument. With the control apparatus 180 coupled at the location 190, actuation of the control apparatus 180 may cause the distal portion 166 to bend at the flexure 164 in the direction D1. The plane (e.g., the X_{A}Y_{A} plane) of the bend angle θ may be consistent or predetermined relative to the sensing element 168 so that with the control apparatus 180 coupled at location 190, the sensing element 168 will predictably bend into generally parallel alignment and abutment with the wall 158. In some examples the bend angle θ may be approximately 30 degrees.

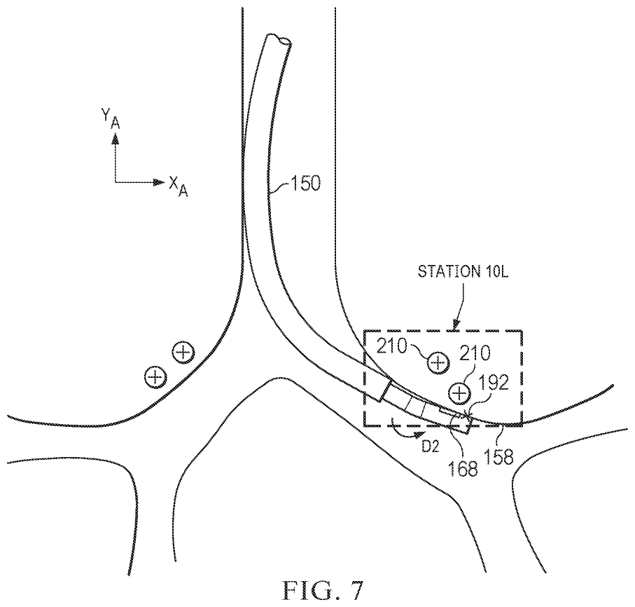

In the example of FIG. 7, a target tissue 210 to be biopsied may include one of a plurality of lymph nodes adjacent to the left bronchial passage that branches from the trachea at regional node station 10L. To access the target tissue 210 in the region of station 10L, the delivery catheter 150 may be positioned against the anatomic wall 158 in the trachea. The distal end portion 151 of the delivery catheter 150 may be steered or bent toward the target tissue 210. The sensing instrument 162 may be extended from the distal end portion 151 with the sensing element 168 oriented in the direction of the target tissue 210. In this example, the control apparatus 180, which may be a single control wire, may be coupled to or terminated at the distal portion 166 or a distal area of the flexure portion 167 of the sensing instrument 162 at a location 192, on the same side as the sensing element 168 or the transmission surface 196 of the sensing instrument. With the control apparatus 180 coupled at the location 192, actuation of the control apparatus 180 may cause the distal portion 166 to bend at the flexure 164 in the direction D2. The plane (e.g., the XY plane) of the bend angle may be consistent or predetermined relative to the sensing element 168 so that with the control apparatus 180 coupled at location 192, the sensing element 168 will predictably bend into generally parallel alignment and abutment with the wall 158.

FIG. 8 illustrates a cross-sectional view of the elongated medical instrument system 160. As shown in the cross-sectional view, the sensing element 168 (e.g., the ultrasound transducer) of the sensing instrument 162 may be recessed beneath the transmission surface 196 (e.g., an ultrasound transmission surface). The control apparatus 180 (e.g., a control wire) may terminate and couple within the distal portion 166 at the location 190 near a distal end of the flexure 164 on a opposite side of the instrument 162 from the transmission surface 196. A flexible jacket 198 may extend around the flexure 164. The passage 174 through which the interventional tool 176 (e.g. a flexible biopsy needle) extends may include a ramp 199 that terminates at the exit port 178, proximally of the flexure 164. As the interventional tool 176 is extended distally through the passage 174, the ramp 199 may guide the flexible needle to emerge from the exit port 178 at a deployment angle φ relative to the longitudinal axis L1 of the sensing instrument 162. In some examples, the angle φ may be between approximately 30 and 45 degrees. In this example, with the exit port 178 proximal of the flexure, the deflection sensor 184 may provide information about the position and/or orientation of the of the articulated sensing element 168 relative to the axis L1 of the sensing instrument 162 and relative to the exit port 178. Thus, information from the deflection sensor 184 may be used to determine the orientation of the deployed needle 176 relative to the sensing element 168.

FIG. 9 illustrates a cross-sectional view of an elongated medical instrument system 250 that may be substantially similar to the medical system 160 with the differences as described. In this example, a sensing instrument 252 includes a sensing element 254 (e.g., an ultrasound transducer) recessed beneath a transmission surface 256 (e.g., an ultrasound transmission surface). In this example, a passage 258 through which the interventional tool 176 (e.g., a flexible biopsy needle) extends may include a ramp 260 that terminates at the exit port 262, distally of a flexure 264. In this example, with the exit port 262 located distally of the flexure 264, the interventional tool 176 may extend through the flexure 264 and bend along with the flexure. Thus, the location and orientation of the exit port 262 and the emergent angle of the interventional tool 176 may remain constant relative to the sensing element 254. This facilitates the interventional tool 176 remaining in the field of view of the sensing element 254 regardless of any bending at the flexure 264

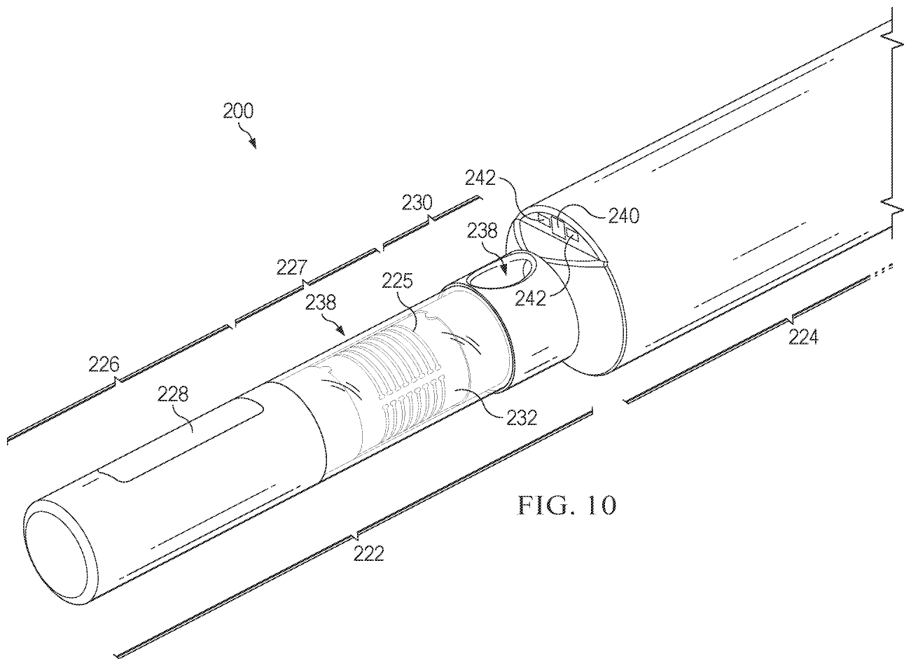

As described above, in some examples the sensing instrument may be insertable through a working channel of a delivery catheter such that the longitudinal motion of the sensing instrument may be independent of the longitudinal motion of the delivery catheter. In other examples, a sensing instrument may be integral with the delivery catheter so that the sensing instrument advances or retracts longitudinally with the movement of the delivery catheter. FIG. 10 illustrates a medical instrument system 220 including a sensing instrument portion 222 fixed to a delivery catheter portion 224. The medical system 220 may be similar to the medical system 160, with integrated features as described. More specifically, the sensing instrument portion 222 may include a flexure portion 227 extending between a distal portion 226 and a proximal portion 230. The flexure portion 227 may include a flexure 225 that allows the distal portion 226 to deflect to engage the passageway wall 158 as previously described. A sensing element 228, which may include an ultrasound transducer, is located at the distal portion 226 of the sensing instrument portion 222. An exit port 238 in the proximal portion 230 of the sensing instrument portion 222 may allow passage of an interventional tool. In this example a flexure control apparatus 232 (e.g., control wires, tendons, or rods) for deflecting the distal portion 226 may extend through the catheter portion 224 and into the sensing instrument portion 222 to the distal portion 226 or to a distal arca of the flexure portion 227. The flexure control apparatus 232 may be separate and independently operated from the cables, linkages, or other steering controls that controllably bend and steer the delivery catheter portion 224. The delivery catheter portion 224 may include motion control and operational features similar to delivery catheter 150, including an integrated optical camera 240 and illumination devices 242 (e.g. light emitting diodes).

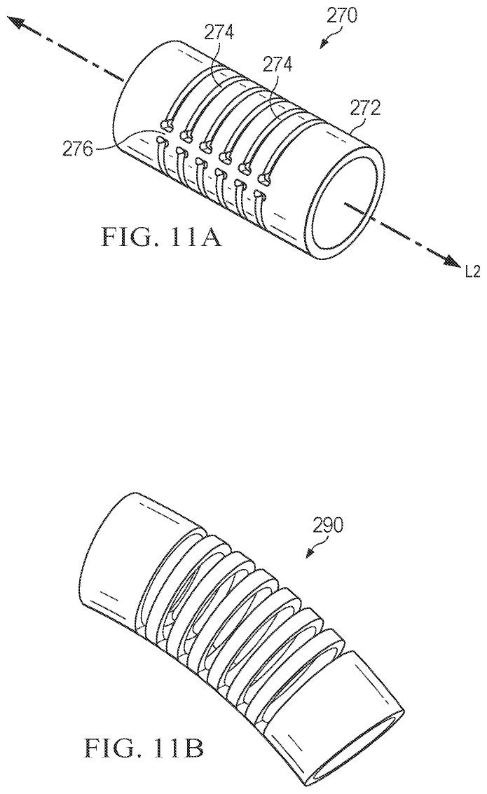

In various examples, the flexures 164, 225, 264 may have any of a variety of configurations that allow the sensing element to pivot or bend relative to a proximal portion of the sensing instrument. For example, the flexure may include a corrugated metal or elastomeric tubular segment, may include a coil spring, or may include a series of links, such as spherical links. FIG. 11A illustrates an example of a flexure 270 that may be formed of a flexible metal (e.g., stainless steel or shape-memory metal such as nitinol), an elastomer, or a polymer. The flexure 270 may include a tubular body 272 with a plurality of slits 274 extending generally perpendicular to a longitudinal axis L2 of the flexure 270. A strut 276 may extend from a distal portion of the tubular body 272 to a proximal portion of the tubular body. When the flexure 270 flexes, the strut 276 may bend and the slits 274 may splay apart to allow for bending of the flexure.

FIG. 11B illustrates an example of a flexure 290 that may be substantially similar to the flexure 270 but in this example, the flexure 290 may be pre-bent to a maximum bend configuration (e.g., when no external forces are exerted) so that the flexure bends repeatedly and consistently in the same plane. Movement of a single control wire (e.g., control apparatus 180) may cause the flexure 290 to extend to a straightened configuration or relax to the pre-bent configuration.

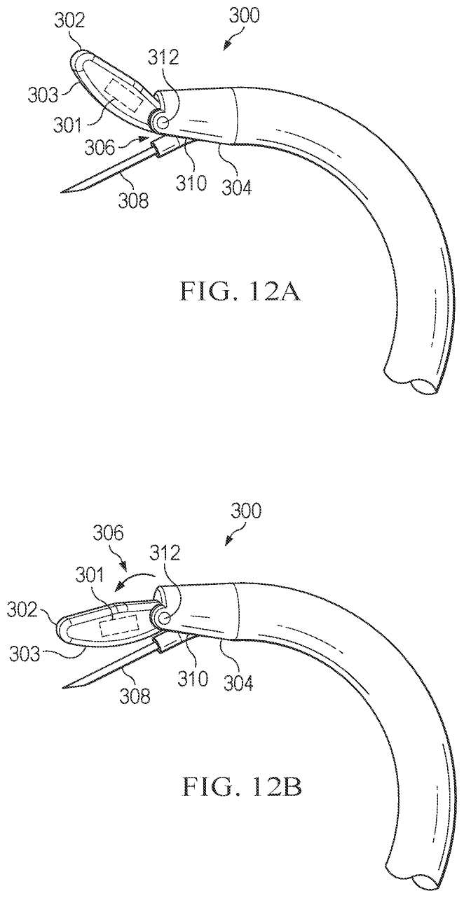

FIGS. 12A and 12B illustrates an example of a sensing instrument 300 including distal portion 302 pivotable relative to a proximal portion 304 by a flexure 306. In this example, the distal portion 302 includes a sensing element 301 (e.g., an ultrasound transducer) recessed beneath a transmission surface 303 (e.g., an ultrasound transmission surface). An interventional tool 308 extends through an exit port 310 in the proximal portion 304. In this example, the flexure 306 may be a hinge joint including a pivot pin 312 connecting the distal portion 302 to the proximal portion 304. The distal portion 302 may be pivoted relative to the instrument shaft by a control apparatus (e.g., control apparatus 180) as previously described.

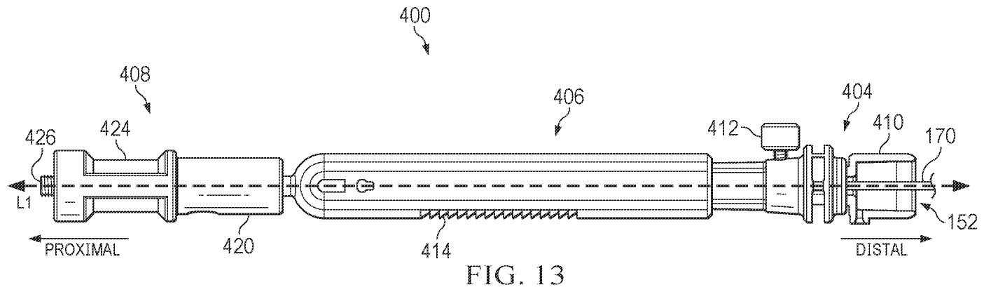

FIG. 13 illustrates a side view of a control apparatus actuation system 400 that may be coupled to the proximal portion 170 of the sensing instrument 162. The control apparatus actuation system 400 may include an instrument connector 404, a handle portion 406, and an actuator device 408. The instrument connector 404 may include a rotatable coupling member 410 and a fixation member 412. The proximal portion 170 of the sensing instrument 162 may be inserted into the coupling member 410. The coupling member 410 may be rotatable to allow the sensing instrument 162 to roll about the axis L1 and to select a roll orientation for the sensing element 168. Once a desired orientation of the sensing element 168 is received, the fixation member 412 (e.g., a set screw) may be tightened to lock the roll orientation of the sensing instrument 162 relative to the handle portion 406. The handle portion 406 may include an elongated grip member 414 that extends proximally of instrument connector 404 and provides a grip surface for an operator. The actuator device 408 extends proximally of the handle portion 406.

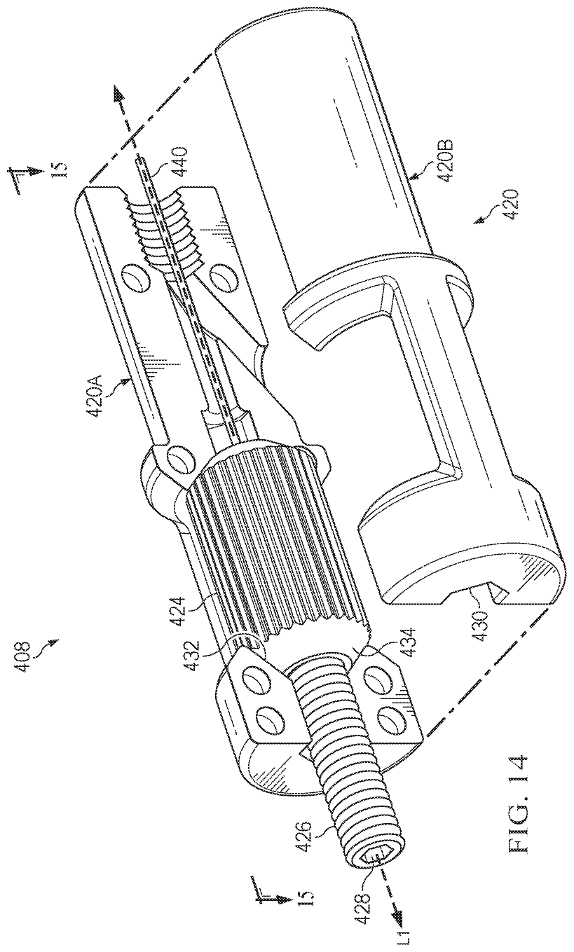

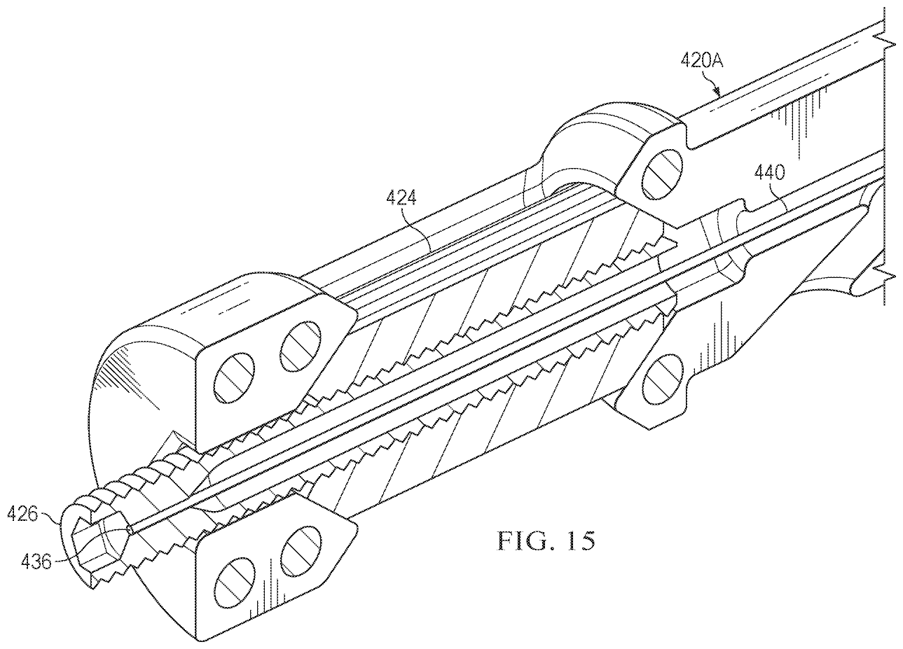

FIG. 14 illustrates a partially exploded view of the actuator device 408 and FIG. 15 illustrates a cross sectional view of the actuator device 408. The actuator device 408 may include a housing 420 including a housing portion 420A and a housing portion 420B that houses a knurled control wheel 424 and a lead screw 426. The lead screw may include external threads that engage internal threads of the knurled control wheel 424. Turning the knurled control wheel 424 about the axis L1 may cause the lead screw to slide axially relative to the housing 420. In this example an exterior hexagonal profile 428 of the lead screw may mate with a hexagonal slot 430 in the housing 420 to prevent the lead screw from rotating. A cone-shaped recess 432 in the housing may mate with cone-shaped ends 434 of the knurled control wheel 424 to prevent the knurled control wheel 424 from moving axially along the axis L1 when the knurled control wheel 424 is rotated. In some examples, the external threads and the internal threads may have a fine pitch sufficient to cause enough friction to stabilize the interaction such that no further locking mechanism is needed to fix the lead screw 426 relative to the knurled control wheel 424. In other examples, a locking mechanism may be included to prevent relative motion.

In this example, a flexure control apparatus 440 (e.g., flexure control apparatus 180) may be a tendon with a proximal end that extends through the lead screw 426 and attaches to the lead screw 426 at an attachment device 436. With the tendon 440 attached to the lead screw 426, linear movement of the lead screw along the axis L1 causes corresponding linear movement of the tendon 440. The linear movement of the tendon 440, which is attached at a distal end to the distal portion 166 of the sensing instrument 162, may cause bending or pivoting of the distal portion 166 at the flexure 164.

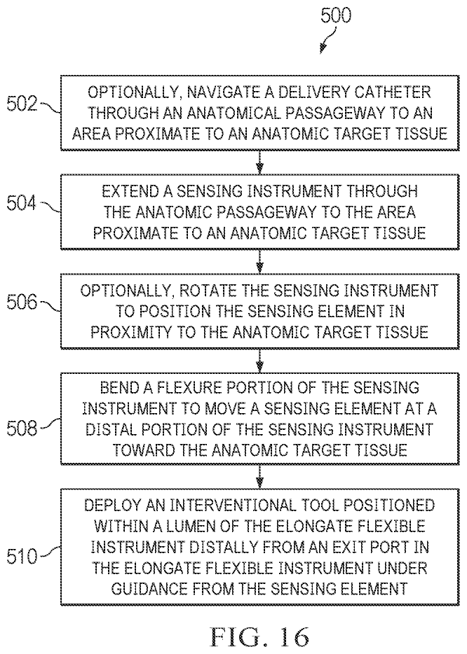

FIG. 16 is a flow chart illustrating a method 500 for deflecting a sensing instrument in a sensor-guided procedure. The method 500 is illustrated as a set of operations or processes that may be performed in the same or in a different order than the order shown. One or more of the illustrated processes may be omitted in some examples of the method. Additionally, one or more processes that are not expressly illustrated in FIG. 16 may be included before, after, in between, or as part of the illustrated processes. In some examples, one or more of the processes of method 500 may be implemented, at least in part, by a control system executing code stored on non-transitory, tangible, machine-readable media that when run by one or more processors (e.g., the processors of a control system) may cause the one or more processors to perform one or more of the processes

At process 502, a delivery catheter may be navigated through an anatomic passageway to an area proximate to an anatomic target tissue. For example, a delivery catheter 150 may be navigated through a passageway 102 to a region of the passageway near a target tissue 113. The target tissue may be, for example, a lesion, nodule, lymph node, or other tissue of interest for investigation or treatment. The target tissue may be external to the anatomic passageway and thus may not be directly viewed with visible light imaging tools. In some examples the delivery catheter may be a manually actuated bronchoscope. In other examples, the delivery catheter may be coupled to a surgical robot and navigated with teleoperational or robotic assistance.

At a process 504, a sensing instrument may be extended through the anatomic passageway to the area proximate to the target tissue. In some examples, the sensing instrument may be extended through a channel in the delivery catheter and distally of a distal end of the delivery catheter. For example, the sensing instrument 162 may be extended through a working channel 156 of the delivery catheter 150 and extended from a distal end portion 151 of the delivery catheter 150. In some examples, the sensing instrument 162 may be deployed without a delivery catheter or may be integral with the delivery catheter as shown in FIG. 10.

At a process 506, the sensing instrument may be rotated (if necessary) to position a sensing element in proximity to the anatomic target tissue. For example, the sensing instrument 162 may be rotated about the axis L1 (e.g., rotate relative to the delivery catheter 150) to position the sensing element 168 in an orientation facing the anatomic passageway wall 158. In some examples, the sensing instrument 162 may be rotated by rotating the coupling member of the control apparatus actuation system 400. When the sensing element 168 is facing the passageway wall 158, further rotation may be prevented by engaging the fixation member 512. In some examples, if the delivery catheter has rotational capability, the delivery catheter and the sensing instrument may be rotated together to achieve the sensor element positioning.

At a process 508, a flexure portion of the sensing instrument is bent to move a sensing element toward the anatomic target tissue. For example, the flexure 164 of sensing instrument 162 may be bent to cause sensing element 168 toward the wall 158 between the target tissue 113 and the sensing instrument 162. The bending of the flexure 164 may change an orientation of the sensing element 168 relative to the proximal portion 170 of the elongate flexible member 161 and may cause the transmission surface 196 to contact the wall 158. In some examples, the flexure 164 may be bent by actuating a control apparatus 180 coupled to the distal portion 166 of the sensing instrument 162. In some examples the actuator device 408 is a component of a control apparatus actuation system 400 that may actuate the flexure 164. For example, the tendon 440 may be coupled between the distal portion 166 and the lead screw 426. As the knurled control wheel 424 is rotated, the lead screw 426 may be advanced or retracted along the axis L1 to move the attached tendon 440. Movement of the tendon 440 may cause bending of the flexure 164 and corresponding movement of the sensing element 168.

In this example, a flexure control apparatus 440 (e.g., flexure control apparatus 180) may be a tendon with a proximal end that extends through the lead screw 426 and attaches to the lead screw 426 at an attachment device 436. With the tendon 440 attached to the lead screw 426, linear movement of the lead screw along the axis L1 causes corresponding linear movement of the tendon 440. The linear movement of the tendon 440, which is attached at a distal end to the distal portion 166 of the sensing instrument 162, may cause bending or pivoting of the distal portion 166 at the flexure 164. If the tendon 440 is coupled to the distal portion 166 on the transmission surface side of the axis L1, the flexure 164 may bend in the direction of the transmission surface side. If the tendon 440 is coupled to the distal portion 166 on an opposite side of the axis L1, the flexure 164 may bend in the direction opposite the transmission surface side. In some examples, a deflection sensor (e.g., the deflection sensor 184) may provide data to measure the deflection of the distal portion 166 or the sensing element 168 relative to the proximal portion 170 of the sensing instrument 162.

At a process 510, an interventional tool positioned within a lumen of the sensing instrument may be deployed distally from an exit port in the elongate flexible instrument under guidance from the sensing element. For example, sensor data such as ultrasound image data, received from an ultrasound transducer (e.g., the sensing element 168) may provide information about the location, size, shape, and/or relative distance from the transducer. Based this image data, the position of the sensing instrument may be adjusted to optimize the deployment of an interventional tool 176, such as a biopsy tool. The tool 176 may be advanced through the passage 174, along the ramp 199, and through the exit port 178. The deployed tool 176 may puncture the wall 158 to engage the target tissue 113 to perform, for example, a biopsy procedure. While the tool 176 is deployed, it may be visible within the field of view of the ultrasound transducer so that trajectory, depth, location, and/or extent of tissue access by the tool 176 may be observed in real time. Optionally, any of the processes 502-510 may be repeated for additional interventional procedures.

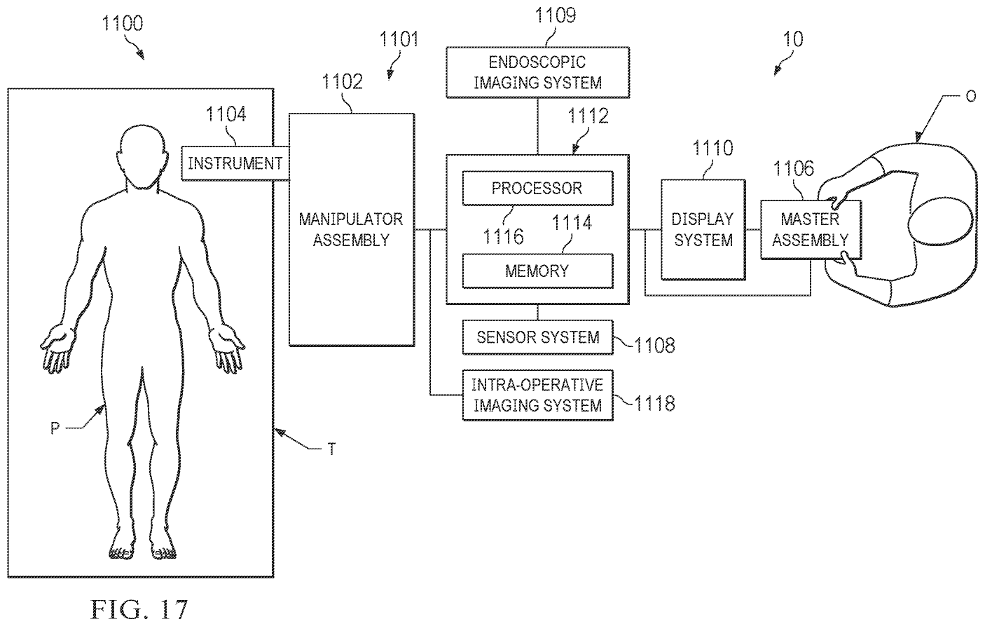

In some examples, medical procedure may be performed using hand-held or otherwise manually controlled imaging probes and tools of this disclosure. In other examples, the described imaging catheters, instruments, and/or tools many be manipulated with a robot-assisted medical system as shown in FIG. 17. FIG. 17 illustrates a robot-assisted medical system 1100. The robot-assisted medical system 1100 generally includes a manipulator assembly 1102 for operating a medical instrument system 1104 (including, for example, medical instrument system 100, 160) in performing various procedures on a patient P positioned on a table T in a surgical environment 1101. The manipulator assembly 1102 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. A master assembly 1106, which may be inside or outside of the surgical environment 1101, generally includes one or more control devices for controlling manipulator assembly 1102. Manipulator assembly 1102 supports medical instrument system 1104 and may include a plurality of actuators or motors that drive inputs on medical instrument system 1104 in response to commands from a control system 1112. The actuators may include drive systems that when coupled to medical instrument system 1104 may advance medical instrument system 1104 into a naturally or surgically created anatomic orifice. Other drive systems may move the distal end of medical instrument system 1104 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulatable end effector of medical instrument system 1104 for grasping tissue in the jaws of a biopsy device and/or the like.

Robot-assisted medical system 1100 also includes a display system 1110 (which may display, for example, an ultrasound image generated by the sensing instrument) for displaying an image or representation of the surgical site and medical instrument system 1104 generated by a sensor system 1108 and/or an endoscopic imaging system 1109. Display system 1110 and master assembly 1106 may be oriented so operator O can control medical instrument system 1104 and master assembly 1106 with the perception of telepresence.

In some examples, medical instrument system 1104 may include components for use in surgery, biopsy, ablation, illumination, irrigation, or suction. In some examples, medical instrument system 1104 may include components of the endoscopic imaging system 1109, which may include an imaging scope assembly or imaging instrument (e.g. a visible light and/or near infrared light imaging) that records a concurrent or real-time image of a surgical site and provides the image to the operator or operator O through the display system 1110. The concurrent image may be, for example, a two or three-dimensional image captured by an imaging instrument positioned within the surgical site. In some examples, the endoscopic imaging system components may be integrally or removably coupled to medical instrument system 1104. However, in some examples, a separate endoscope, attached to a separate manipulator assembly may be used with medical instrument system 1104 to image the surgical site. The endoscopic imaging system 1109 may be implemented as hardware, firmware, software, or a combination thereof which interact with or are otherwise executed by one or more computer processors, which may include the processors of the control system 1112.

The sensor system 1108 may include a position/location sensor system (e.g., an electromagnetic (EM) sensor system) and/or a shape sensor system for determining the position, orientation, speed, velocity, pose, and/or shape of the medical instrument system 1104.

Robot-assisted medical system 1100 may also include control system 1112. Control system 1112 includes at least one memory 1116 and at least one computer processor 1114 for effecting control between medical instrument system 1104, master assembly 1106, sensor system 1108, endoscopic imaging system 1109, intra-operative imaging system 1118, and display system 1110. Control system 1112 (which may include a controller in operative communication with the imaging device 111) also includes programmed instructions (e.g., a non-transitory machine-readable medium storing the instructions) to implement some or all of the methods described in accordance with aspects disclosed herein, including instructions for providing information to display system 1110.

Control system 1112 may further include a virtual visualization system to provide navigation assistance to operator O when controlling medical instrument system 1104 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

An intra-operative imaging system 1118 may be arranged in the surgical environment 1101 near the patient P to obtain images of the anatomy of the patient P during a medical procedure. The intra-operative imaging system 1118 may provide real-time or near real-time images of the patient P. In some examples, the intra-operative imaging system 1118 may comprise an ultrasound imaging system for generating two-dimensional and/or three-dimensional images. For example, the intra-operative imaging system 1118 may be at least partially incorporated into sensing instrument 162. In this regard, the intra-operative imaging system 1118 may be partially or fully incorporated into the medical instrument system 1104.

FIG. 18A is a simplified diagram of a medical instrument system 900 configured in accordance with various embodiments of the present technology. The medical instrument system 900 includes an elongate flexible device 902 (e.g., delivery device 150), such as a flexible catheter, coupled to a drive unit 904. The elongate flexible device 902 includes a flexible body 916 having a proximal end 917 and a distal end or tip portion 918. The medical instrument system 900 further includes a tracking system 930 for determining the position, orientation, speed, velocity, pose, and/or shape of the distal end 918 and/or of one or more segments 924 along the flexible body 916 using one or more sensors and/or imaging devices as described in further detail below.

The tracking system 930 may optionally track the distal end 918 and/or one or more of the segments 924 using a shape sensor 922. The shape sensor 922 may optionally include an optical fiber aligned with the flexible body 916 (e.g., provided within an interior channel (not shown) or mounted externally). The optical fiber of the shape sensor 922 forms a fiber optic bend sensor for determining the shape of the flexible body 916. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. Pat. No. 7,781,724 (filed Sep. 26, 2006, disclosing “Fiber optic position and shape sensing device and method relating thereto”; U.S. Pat. No. 7,772,541, filed Mar. 12, 2008, titled “Fiber Optic Position and/or Shape Sensing Based on Rayleigh Scatter”; and U.S. Pat. No. 6,389,187, filed Apr. 21, 2000, disclosing “Optical Fiber Bend Sensor,” which are all incorporated by reference herein in their entireties. In some embodiments, the tracking system 930 may optionally and/or additionally track the distal end 918 using a position sensor system 920. The position sensor system 920 may be a component of an EM sensor system with the position sensor system 920 including one or more conductive coils that may be subjected to an externally generated electromagnetic field. In some embodiments, the position sensor system 920 may be configured and positioned to measure six degrees of freedom (e.g., three position coordinates X, Y, and Z and three orientation angles indicating pitch, yaw, and roll of a base point) or five degrees of freedom (e.g., three position coordinates X, Y, and Z and two orientation angles indicating pitch and yaw of a base point). Further description of a position sensor system is provided in U.S. Pat. No. 6,380,732, filed Aug. 9, 1999, disclosing “Six-Degree of Freedom Tracking System Having a Passive Transponder on the Object Being Tracked,” which is incorporated by reference herein in its entirety. In some embodiments, an optical fiber sensor may be used to measure temperature or force. In some embodiments, a temperature sensor, a force sensor, an impedance sensor, or other types of sensors may be included within the flexible body. In various embodiments, one or more position sensors (e.g., fiber shape sensors, EM sensors, and/or the like) may be integrated within the medical instrument 926 and used to track the position, orientation, speed, velocity, pose, and/or shape of a distal end or portion of medical instrument 926 using the tracking system 930.

The flexible body 916 includes a channel 921 sized and shaped to receive a medical instrument 926 (e.g., sensing instrument 152, 162, 252, 300). FIG. 18B, for example, is a simplified diagram of the flexible body 916 with the medical instrument 926 extended according to some embodiments. In some embodiments, the medical instrument 926 may be used for procedures such as imaging, visualization, surgery, biopsy, ablation, illumination, irrigation, and/or suction. The medical instrument 926 can be deployed through the channel 921 of the flexible body 916 and used at a target location within the anatomy. The medical instrument 926 may include, for example, image capture probes, biopsy instruments, ablation needles, electroporation needles, laser ablation fibers, and/or other surgical, diagnostic, or therapeutic tools, including any of the instrument systems described above. The medical instrument 926 may be advanced from the opening of channel 921 to perform the procedure and then be retracted back into the channel 921 when the procedure is complete. The medical instrument 926 may be removed from the proximal end 917 of the flexible body 916 or from another optional instrument port (not shown) along the flexible body 916.

In some examples, an optical or visible light imaging instrument (e.g., an image capture probe) may extend within the channel 921 or within the structure of the flexible body 916. The imaging instrument may include a cable coupled to the camera for transmitting the captured image data. In some embodiments, the imaging instrument may be a fiber-optic bundle, such as a fiberscope, that couples to an image processing system 931. The imaging instrument may be single or multi-spectral, for example capturing image data in one or more of the visible, infrared, and/or ultraviolet spectrums.

The flexible body 916 may also house cables, linkages, or other steering controls (not shown) that extend between the drive unit 904 and the distal end 918 to controllably bend the distal end 918 as shown, for example, by broken dashed line depictions 919 of the distal end 918. In some embodiments, at least four cables are used to provide independent “up-down” steering to control a pitch of the distal end 918 and “left-right” steering to control a yaw of the distal end 918. Steerable elongate flexible devices are described in detail in U.S. Pat. No. 9,452,276, filed Oct. 14, 2011, disclosing “Catheter with Removable Vision Probe,” and which is incorporated by reference herein in its entirety. In various embodiments, medical instrument 926 may be coupled to drive unit 904 or a separate second drive unit (not shown) and be controllably or robotically bendable using steering controls.

The information from the tracking system 930 may be sent to a navigation system 932 where it is combined with information from the image processing system 931 and/or the preoperatively obtained models to provide the operator with real-time position information. In some embodiments, the real-time position information may be displayed on the display system 1110 of FIG. 17 for use in the control of the medical instrument system 900. In some embodiments, the control system 1112 of FIG. 17 may utilize the position information as feedback for positioning the medical instrument system 900. Various systems for using fiber optic sensors to register and display a surgical instrument with surgical images are provided in U.S. Pat. No. 8,900,131, filed May 13, 2011, disclosing “Medical System Providing Dynamic Registration of a Model of an Anatomic Structure for Image-Guided Surgery,” which is incorporated by reference herein in its entirety.

In some embodiments, the medical instrument system 900 may be teleoperated or robot-assisted within the medical system 1100 of FIG. 17. In some embodiments, the manipulator assembly 1102 of FIG. 17 may be replaced by direct operator control. In some embodiments, the direct operator control may include various handles and operator interfaces for hand-held operation of the instrument.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

The methods described herein are illustrated as a set of operations or processes. Not all the illustrated processes may be performed in all examples of the methods. Additionally, one or more processes that are not expressly illustrated or described may be included before, after, in between, or as part of the example processes. In some examples, one or more of the processes may be performed by the control system (e.g., control system 1112) or may be implemented, at least in part, in the form of executable code stored on non-transitory, tangible, machine-readable media that when run by one or more processors (e.g., the processors 1114 of control system 1112) may cause the one or more processors to perform one or more of the processes.

One or more elements in examples of this disclosure may be implemented in software to execute on a processor of a computer system such as control processing system. When implemented in software, the elements of the examples of the invention are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In one example, the control system supports wireless communication protocols such as Bluetooth, IrDA, HomeRF, IEEE 802.11, DECT, and Wireless Telemetry.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

In some instances well known methods, procedures, components, and circuits have not been described in detail so as not to unnecessarily obscure aspects of the examples. This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

While certain exemplary examples of the invention have been described and shown in the accompanying drawings, it is to be understood that such examples are merely illustrative of and not restrictive on the broad invention, and that the examples of the invention not be limited to the specific constructions and arrangements shown and described, since various other modifications may occur to those ordinarily skilled in the art.

What is claimed is:

1. A system comprising:
a sensing instrument, including:
an elongate flexible member including a channel extending therein, the elongate flexible member including a proximal portion, a distal portion, and a flexure portion between the distal and proximal portions;
a flexure at the flexure portion of the elongate flexible member;
a sensing element coupled at the distal portion of the elongate flexible member;
a flexure control apparatus extending within the elongate flexible member and configured to bend the flexure to change an orientation of the sensing element relative to the proximal portion of the elongate flexible member; and
an exit port in the proximal portion of the elongate flexible member, the exit port extending between the channel and an outer surface of the elongate flexible member.

2. The system of claim 1, further comprising an interventional tool configured to extend through the channel and exit the elongate flexible member through the exit port.

3. The system of claim 2, wherein the interventional tool includes a biopsy needle.

4. The system of claim 2, wherein the interventional tool includes an ablation tool or electroporation tool.

5. The system of claim 2, wherein the interventional tool is configured to deliver a device into a patient anatomy.

6. The system of claim 1, wherein the sensing element includes an imaging element.

7. The system of claim 6, wherein the imaging element includes an ultrasound transducer.

8. The system of claim 6, wherein the imaging element includes a visible light camera.

9. The system of claim 1, wherein the sensing element includes an infrared sensor.

10. The system of claim 1, wherein the flexure includes a shape memory tube, and elastomeric tube, or a polymer tube.

11. The system of claim 1, wherein the flexure includes a plurality of slits, a plurality of links, or a hinge.

12. The system of claim 1, wherein the flexure control apparatus includes a push-pull wire.

13. The system of claim 1, wherein the flexure control apparatus includes a plurality of tendons.

14. The system of claim 1, wherein the flexure control apparatus is configured to be moved by a robot-assisted manipulator.

15. The system of claim 1, wherein the flexure control apparatus is configured for manual control.

16. The system of claim 1, wherein the sensing instrument further includes a deflection sensor.

17. The system of claim 16, wherein the deflection sensor includes an optical fiber shape sensor.

18. The system of claim 1, further comprising a delivery catheter including a channel configured to receive the sensing instrument, the delivery catheter configured to articulate independently of bending of the flexure.

19. The system of claim 18, wherein the proximal portion of the elongate flexible member is configured to articulate independently of the bending of the flexure.

20. The system of claim 1, further comprising a delivery catheter portion, wherein the sensing instrument is coupled to a distal end of the delivery catheter portion and bending of the flexure is controllable independently of bending of the delivery catheter portion.

21. A method comprising:
positioning a sensing instrument in an anatomic passageway proximate to an anatomic target tissue, the sensing instrument including an elongate flexible member, a flexure, a sensing element coupled at a distal portion of the elongate flexible member, a flexure control apparatus extending within the elongate flexible member, and an exit port in a proximal portion of the elongate flexible member;
bending the sensing instrument at the flexure with the flexure control apparatus to move the sensing element toward the anatomic target tissue; and
deploying an interventional tool through the elongate flexible member and through the exit port under guidance from the sensing element.

22. The method of claim 21, further comprising:
rotating the sensing element to position the sensing element toward the anatomic target tissue.

23. The method of claim 21, further comprising:
receiving the guidance from the sensing element, wherein the guidance from the sensing element includes ultrasound image data from the sensing element; and
adjusting the sensing instrument based on the guidance.
